(19) United States
(12) Patent Application Publication
(10) Pub. No.: US 2005/0215604 A1
(43) Pub. Date: Sep. 29, 2005

(54) Combination therapies with epothilones and carboplatin

(75) Inventors: Yiqing Zhou, Lafayette, CA (US); Robert G. Johnson JR., Lafayette, CA (US)

Correspondence Address: KOSAN BIOSCIENCES, INC., 3832 BAY CENTER PLACE, HAYWARD, CA, 94545, US

(73) Assignee: Kosan Biosciences, Inc., Hayward, CA (US)

(21) Appl. No.: 11/088,534

(22) Filed: Mar. 23, 2005

Related U.S. Application Data
(60) Provisional application No. 60/556,856, filed on Mar. 26, 2004.

(51) Int. Cl. A61K 31/427; A61K 31/28

(52) U.S. Cl. 514/365; 514/492

(57) ABSTRACT

A combination therapy of an epothilone and carboplatin is effective for treating tumors.

### CROSS-REFERENCE TO RELATED APPLICATIONS

This application claims the benefit of U.S. Provisional Application No. 60/556,856 filed Mar. 26, 2004; the disclosure of which is incorporated herein by reference.

### BACKGROUND OF THE INVENTION

1. Field of the Invention

This invention relates to combination therapies with epothilones and carboplatin and methods for their administration.

2. Description of Related Art

The epothilones are potent stabilizers of microtubule formation with a mechanism of action similar to that of paclitaxel: inhibition of tubulin depolymerization and blockage at G2/M of the cell cycle. Of particular interest are epothilone B (1) and its derivatives, for example epothilone B lactam and 21-aminoepothilone B, and epothilone D (2, also known as “KOS-862”) and its derivatives.

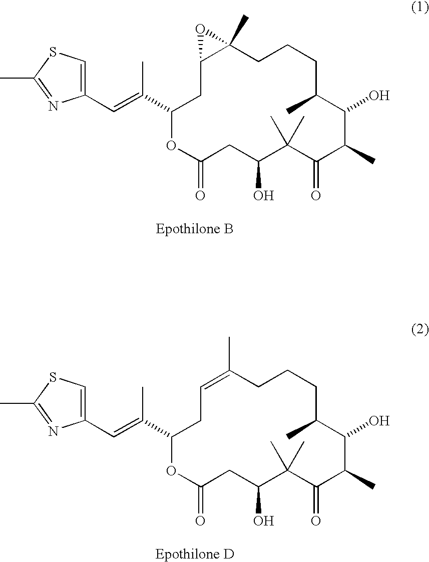

Unlike paclitaxel, epothilone D maintains its potency against paclitaxel resistant human cancer cell lines both in vitro and in vivo. Epothilone D has entered multiple monotherapy Phase 2 clinical trials and shows significant promise in the treatment of various cancers.

Combination therapy is important in cancer treatment, as the combination of agents having different mechanisms of action may lead to enhanced cytotoxicity. Since the epothilones (microtubule stabilization) and carboplatin (DNA alkylation) have different mechanisms of antitumor activity, there is potential for non-overlapping toxicities and improved efficacy of an epothilone/carboplatin combination over a taxane/carboplatin combination. The present invention provides combination therapies using epothilone D and carboplatin (Paraplatin™, Bristol-Myers Squibb) that are expected to show significantly improved treatments for various cancers and diseases characterized by cellular hyperproliferation.

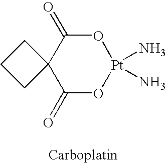

### BRIEF SUMMARY OF THE INVENTION

The invention provides methods for treating diseases characterized by cellular hyperproliferation comprising administering to a patient in need of such treatment a combination of an epothilone and carboplatin. In a preferred embodiment, the epothilone is epothilone D. In another preferred embodiment, the disease of cellular hyperproliferation is cancer, in particular non-small cell lung cancer. In preferred embodiments, the epothilone is administered before or simultaneously with the carboplatin.

In another embodiment, there is provided a pharmaceutical composition comprising an epothilone together with carboplatin and a pharmaceutically acceptable carrier. The carrier can be, for example, water for injection (WFI).

In another embodiment, there is provided a kit comprising a first pharmaceutical composition comprising an epothilone and a second pharmaceutical composition comprising carboplatin.

### BRIEF DESCRIPTION OF THE DRAWING(S)

FIG. 1 shows the data for in vitro tests of combinations epothilone and carboplatin.

FIGS. 2a and 2b show mouse xenograft data for epothilone-carboplatin combination treatment, compared to either agent alone.

FIG. 3 show cell cycle data for epothilone-carboplatin combination treatment, compared to either agent alone.

### DETAILED DESCRIPTION OF THE INVENTION

In one aspect, the invention provides methods for treating diseases characterized by cellular hyperproliferation comprising administering to a patient (in particular a human) in need of such treatment a combination of an epothilone and carboplatin.

The epothilone used in the pharmaceutical compositions of the invention can be any epothilone, and, more particularly, any epothilone having useful therapeutic properties. Such epothilones can be obtained using any combination of total chemical synthesis, partial chemical synthesis, or chemobiosynthesis methods and materials known to those of skill in organic chemistry, medicinal chemistry, and biotechnology arts. Specific examples of epothilones having useful therapeutic properties include, but are not limited to, epothilone A, epothilone B, epothilone C, epothilone D, 4-desmethyl-epothilone D, epothilone B lactam, 21-aminoepothilone B, 9, 10-dehydroepothilone D, 9, 10-dehydro-26-trifluoro-epothilone D, 11 -hydroxyepothilone D, 19-oxazolyl-epothilone D, 10, 11-dehydro-epothilone D, and 19-oxazolyl-10, 11-dehydro-epothilone D. In particular embodiments of the invention, the epothilone is selected from the group consisting of epothilone B, epothilone B lactam, 21-aminoepothilone B, epothilone D, and trans-9,10-dehydroepothilone D. Many detailed examples of suitable epothilones together with methods for their preparation have been published in the literature, with which the skilled practitioner will be familiar.

In certain embodiments of the invention, the disease is cancer. The present invention includes methods for treating cancers of the head and neck which include tumors of the head, neck, nasal cavity, paranasal sinuses, nasopharynx, oral cavity, oropharynx, larynx, hypopharynx, salivary glands, and paragangliomas; cancers of the liver and biliary tree, particularly hepatocellular carcinoma; intestinal cancers, particularly colorectal cancer; treat ovarian cancer; small cell and non-small cell lung cancer; breast cancer sarcomas, such as fibrosarcoma, malignant fibrous histiocytoma, embryonal rhabdomysocarcoma, leiomysosarcoma, neurofibrosarcoma, osteosarcoma, synovial sarcoma, liposarcoma, and alveolar soft part sarcoma; neoplasms of the central nervous systems, particularly brain cancer; lymphomas such as Hodgkin's lymphoma, lymphoplasmacytoid lymphoma, follicular lymphoma, mucosa-associated lymphoid tissue lymphoma, mantle cell lymphoma, B-lineage large cell lymphoma, Burkitt's lymphoma, and T-cell anaplastic large cell lymphoma, among others. In particular embodiments of the invention, the disease is a cancer selected from the group consisting of breast, colorectal, and non-small cell lung cancers.

Clinically, practice of the methods and use of compositions described herein will result in a reduction in the size or number of the cancerous growth and/or a reduction in associated symptoms (where applicable). Pathologically, practice of the method and use of compositions described herein will produce a pathologically relevant response, such as: inhibition of cancer cell proliferation, reduction in the size of the cancer or tumor, prevention of further metastasis, and inhibition of tumor angiogenesis. The method of treating such diseases comprises administering a therapeutically effective amount of an inventive combination to a subject. The method may be repeated as necessary.

In another aspect, the invention provides methods for treating disease comprising administering the combinations described above in certain dosing regimens, described herein. The epothilone can be administered simultaneously with carboplatin. Alternatively, the epothilone can be administered prior to administration of carboplatin, or carboplatin may be administered before the epothilone. In addition, for those embodiments in which the two agents are administered separately, the administration of the second agent can be delayed to provide greater therapeutic effect of the combination therapy. Those having skill in the pharmacology and medicine arts will be familiar with concepts, methods, and materials suitable to determine the temporal factors in administering non-simultaneous therapies. Example of relevant factor may include, but are not limited to, the patient's circadian rhythm, cell cycle characteristics relevant to the disease being treated (e.g., tumor cell type), and the pharmacokinetic parameters of the drugs being used.

In another aspect, the invention provides pharmaceutical compositions comprising an epothilone together with carboplatin and a pharmaceutically acceptable carrier. The epothilone and the carboplatin are provided in the appropriate ratio to provide the effective therapeutic doses of the two agents.

In another aspect, the invention provides kits that facilitate the combination therapy using the epothilone and carboplatin as separate agents. In one embodiment, such kits comprise separate pharmaceutical compositions for the two agents, that is, a first pharmaceutical composition comprising an epothilone and a second pharmaceutical composition comprising carboplatin. These pharmaceutical compositions may be concentrates or lyophilates comprising the epothilone or the carboplatin in appropriate amounts together with required excipients, ready for dilution with an aqueous medium prior to injection into the patient, or they may be lipid emulsions ready for direct injection into the patient.

The following Examples merely illustrate certain aspects of the present invention to aid those of skill in the art in practicing the invention, and do not limit the scope of the invention in any manner.

### EXAMPLE 1

### Synergy Between Epothilone D and Carboplatin in Cultured NSCLC Cell Lines

The human NSCLC (non-small cell lung cancer) cell lines A549, H23, H226, H358, H460, and H522 were seeded in 96 well plates (5000 cells/well); after overnight incubation, the cells were treated with epothilone D or carboplatin alone or epothilone D and carboplatin in combination. Once the IC₅₀ value of each drug was obtained, the combined drug treatment was designed at constant ratios of the two drugs and the treatment schedule varied: either epothilone D or carboplatin was administered first with the second drug added 24 hours later. Cell viability was assayed by the MTS assay. While mild antagonism was observed when carboplatin was administered first, marked synergy was measured when epothilone D was initially administered followed by the carboplatin in all cell lines tested. As shown in Table 1, epothilone D shows potent cytotoxic effects against a range of cultured non-small cell lung cancer (NSCLC) human cancer cell lines, whereas carboplatin is essentially inactive at a concentration of 10,000 nM. Cells were treated with epothilone D or carboplatin at varying concentrations, ranging from 1 pM to 10 μM, for 3 days. Cell viability was determined using the using the MTS assay (Promega). IC₅₀ is defined as the concentration of drug required to inhibit cell growth by 50%.

TABLE 1
Activity of Epothilone or Carboplatin Alone against
Human NSCLC Cell Lines

| Cell Line | Epothilone D (IC₅₀, nM) | Carboplatin (IC₅₀, nM) |
|---|---|---|
| A549 | 40 | >10,000 |
| H23 | 37 | >10,000 |
| H226 | 50 | >10,000 |
| H358 | 40 | >10,000 |
| H460 | 33 | >10,000 |
| H522 | 40 | >10,000 |

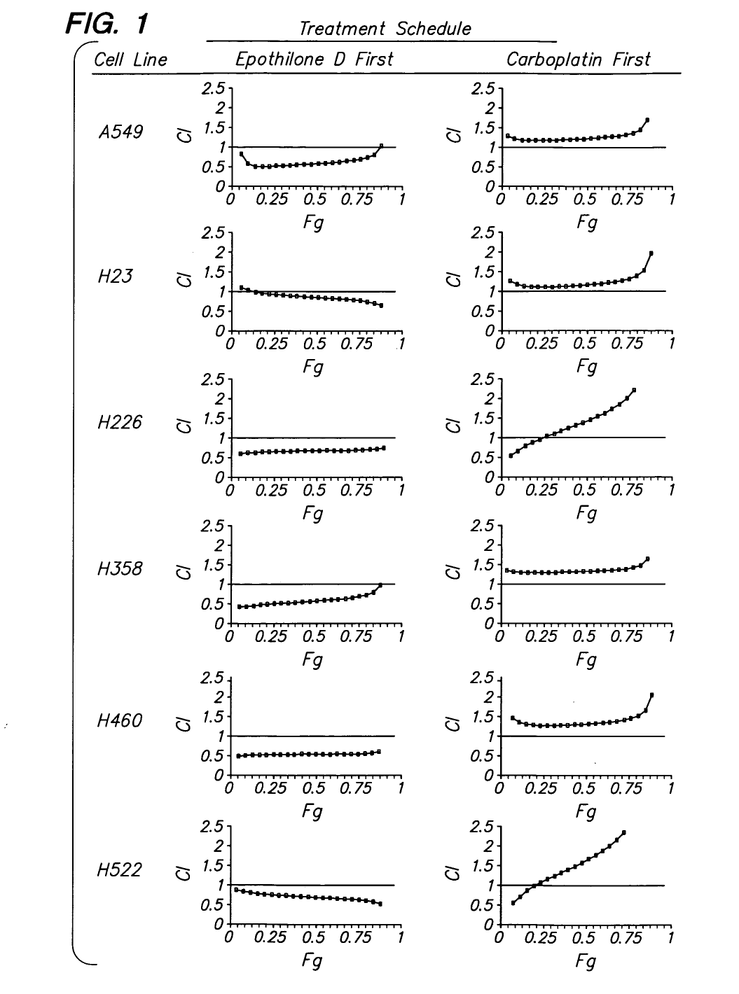

FIG. 1 shows the results of the combination experiments. In all six cell lines, treatment with epothilone D first followed by carboplatin resulted in strong synergy, whereas treatment with carboplatin first followed by epothilone D resulted in mild antagonism. Since carboplatin is shown in Table 1 to be essentially inactive against NSCLC cell lines, the occurrence of synergism in a combination therapy with an epothilone is unexpected.

### EXAMPLE 2

### Synergy Between Epothilone D and Carboplatin in Mouse Xenografts

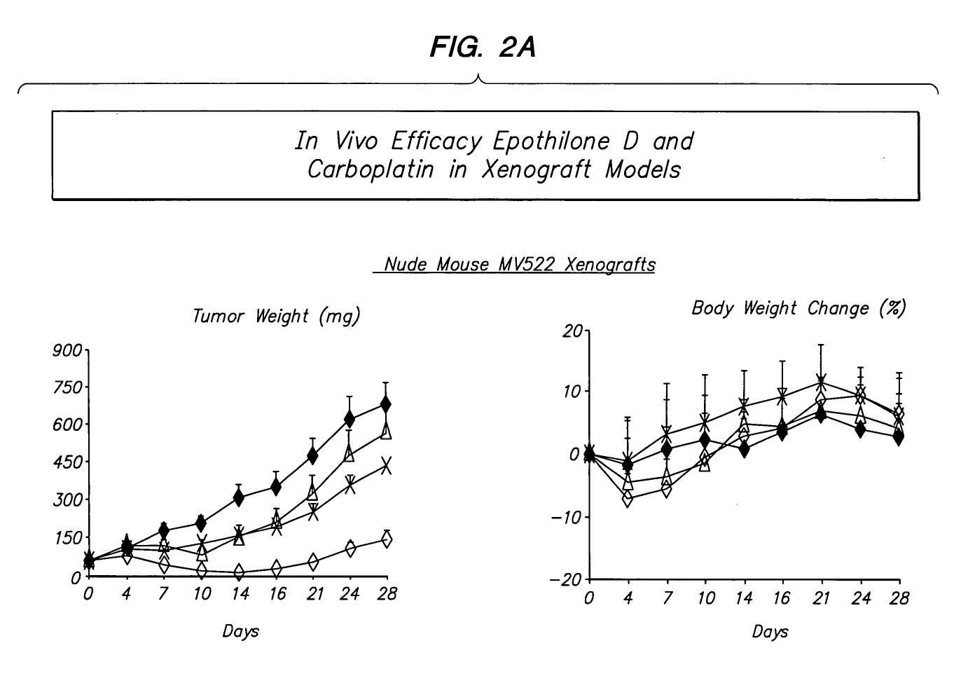

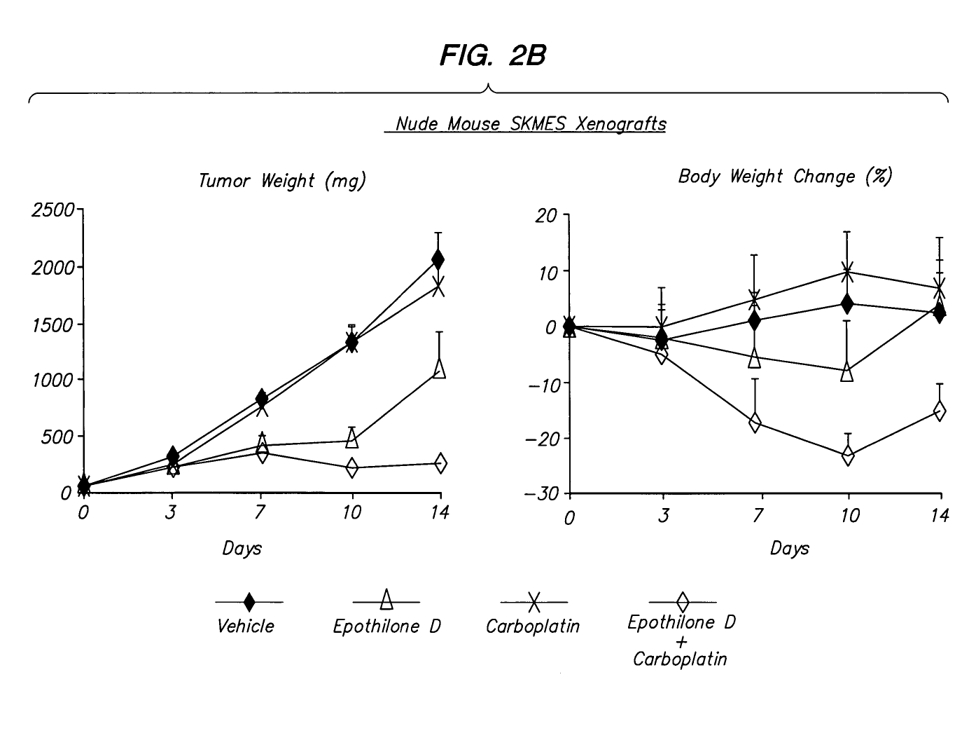

The drug combination was also tested in nude mice bearing the human lung cancer xenografts MV522 and SK-MES. Fragments of human tumor carcinomas harvested from subcutaneously growing tumors in nude mice hosts were implanted subcutaneously. When tumors were approximately 60-75 mg in size (10-14 days after implantation), mice were pair matched into treatment and control groups. Epothilone D was given intraperito-neally twice a day for 7 days (6 to 14 mg/kg/day), carboplatin intraperitoneally every daily for 5 days (10 to 40 mg/kg/day), various combinations of epothilone D (first) and carboplatin (second), or vehicle control. Epothilone D and carboplatin as single agents exerted antiproliferative activity (measured as tumor size) in a dose dependent manner. None of the drug combinations resulted in antagonism while multiple combinations demonstrated additive or synergistic effects under conditions where weight loss of the animals was well tolerated and reversible. The results on an experiment wherein the animals were treated with vehicle, epothilone D (8 mg/kg for MV522 and 14 mg/kg for SKMES, twice a day for 7 days) alone, or carboplatin (10 mg/kg, once daily, for 5 days) alone, or epothilone D followed by carboplatin, are shown in FIG. 2. In the MV522 and SKMES xenograft models, the combination therapy of epothilone D and carboplatin produced a strong tumor inhibition at doses that were significantly less than for single-agent therapy of either drug. The total body weight loss of the animals was well tolerated and reversible.

### EXAMPLE 3

### Effects of Epothilone D and Carboplatin on Cell Cycles

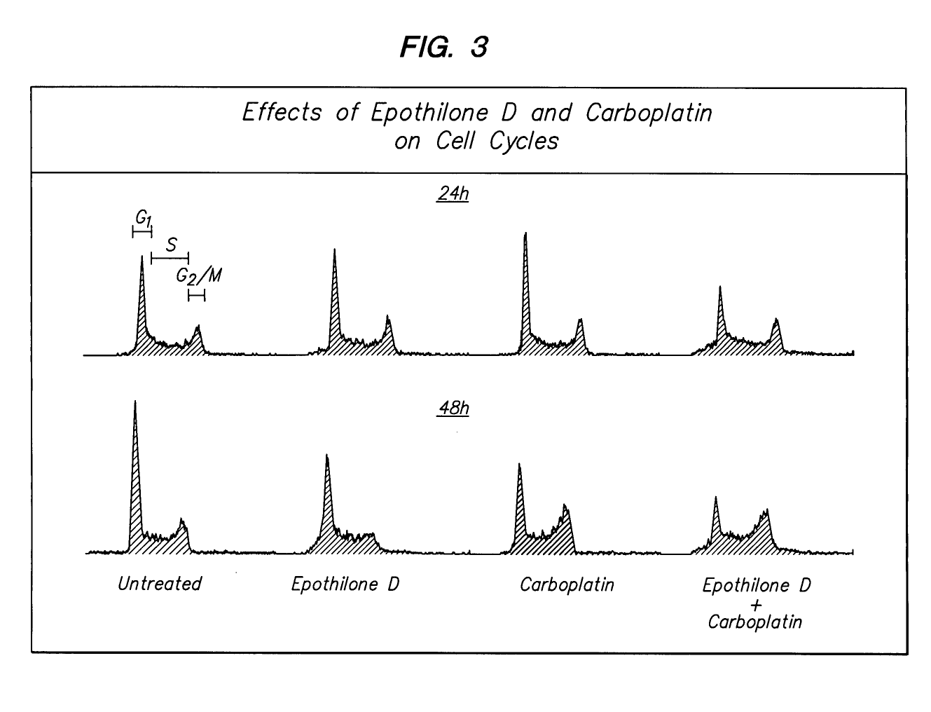

Cells were treated with epothilone D alone (8 nM), carboplatin alone (15 uM), or with epothilone D (8 nM) followed by carboplatin (15 uM). Cells were stained with propidium iodide and analyzed by flow cytometry. Results are shown in FIG. 3. Treatment of cells with a combination of epothilone D and carboplatin results in an increased sub-G₁ population, indicating the induction of apoptosis.

The foregoing detailed description of the invention includes passages that are chiefly or exclusively concerned with particular parts or aspects of the invention. It is to be understood that this is for clarity and convenience, that a particular feature may be relevant in more than just the passage in which it is disclosed, and that the disclosure herein includes all the appropriate combinations of information found in the different passages. Similarly, although the various figures and descriptions herein relate to specific embodiments of the invention, it is to be understood that where a specific feature is disclosed in the context of a particular figure or embodiment, such feature can also be used, to the extent appropriate, in the context of another figure or embodiment, in combination with another feature, or in the invention in general.

Further, while the present invention has been particularly described in terms of certain preferred embodiments, the invention is not limited to such preferred embodiments. Rather, the scope of the invention is defined by the appended claims.

1. A method for treating diseases characterized by cellular hyperproliferation, comprising administering to a patient in need of such treatment a combination of an epothilone and carboplatin.

2. The method of claim 1, wherein the epothilone is epothilone D.

3. The method of claim 2, wherein the disease is cancer.

4. The method of claim 3, wherein the disease is non-small cell lung cancer.

5. The method of claim 1, wherein the disease is cancer.

6. The method of claim 5, wherein the disease is non-small cell lung cancer.

7. The method of claim 1, wherein the patient is first treated with the epothilone, and subsequently is treated with carboplatin.

8. The method of claim 1, wherein the patient is treated simultaneously with the epothilone and with carboplatin.

9. A pharmaceutical composition comprising an epothilone together with carboplatin and a pharmaceutically acceptable carrier.

10. The pharmaceutical composition of claim 9, wherein the epothilone is epothilone D.

11. A kit comprising a first pharmaceutical composition comprising an epothilone and a second pharmaceutical composition comprising carboplatin.

12. The kit of claim 11, wherein the epothilone is epothilone D.
